(19) United States
(12) Patent Application Publication
(10) Pub. No.: US 2015/0329601 A1
(43) Pub. Date: Nov. 19, 2015

(54) Methoxypolyethyleneglycol succinimidyl propionate modified recombinant Ganoderma Lucidum immunoregulatory protein, preparing method and application thereof

(71) Applicants: Xitian Zhang, Shanghai (CN); Fei Sun, Shanghai (CN)

(72) Inventors: Xitian Zhang, Shanghai (CN); Fei Sun, Shanghai (CN); Chongyang Liang, Shanghai (CN)

(73) Assignees: Xitian Zhang; Fei Sun

(21) Appl. No.: 14/814,356

(22) Filed: Jul. 30, 2015

Related U.S. Application Data
(63) Continuation-in-part of application No. 14/119,042, filed on Nov. 20, 2013.

(51) Int. Cl. C07K 14/37

(52) U.S. Cl. CPC C07K 14/37; A61K 38/00

(57) ABSTRACT

Methoxypolyethyleneglycol succinimidyl propionate modified recombinant Ganoderma Lucidum immunoregulatory protein, a preparing method and applications thereof are provided, including: the mPEG-SPA modified rLZ-8; the method for preparing the mPEG-SPA modified rLZ-8 comprising: feeding the rLZ-8 dimer and the mPEG-SPA with the molar ratio of 1:1-1:6 into a 0.1M phosphate buffer with pH 5.0-pH 8.0, and stirring by a magnetic stirrer at a room temperature for 1.0-2.5 h, purifying the product for obtaining a modification product with a purity of 98%; and applications of the mPEG-SPA modified rLZ-8 in preparation of medicine for treating leukopenia due to chemotherapy. Advantages are as follows: the method for preparing the mPEG-SPA modified rLZ-8 is simple, and the product is identical; a half-life of the mPEG-SPA modified rLZ-8 is significantly longer than the half-life of unmodified rLZ-8 (illustrated in the FIG. 2); a minimum effective dosage and time for treating leucopenia are also improved.

### CROSS REFERENCE OF RELATED APPLICATION

This is a Continuation-In-Parts application of the International Application PCT/CN2013/076665, filed Jun. 3, 2013, which claims priority under 35 U.S.C. 119(a-d) to CN 201210243582.7, filed Jul. 16, 2012.

### BACKGROUND OF THE PRESENT INVENTION

1. Field of Invention

The present invention relates to modifying protein with methoxypolyethyleneglycol succinimidyl propionate, and more particularly to methoxypolyethyleneglycol succinimidyl propionate modified recombinant Ganoderma Lucidum immunoregulatory protein rLZ-8 and a preparing method thereof.

2. Description of Related Arts

Chemotherapy is one of the effective methods for treating cancer, but chemotherapy drugs often cause decrease in the number of white blood cells (WBCs), which lead to leucopenia. Leucopenia is characterized by decrease in the total number of WBCs, which are usually diagnosed by the count of white blood cells less than 4.0×10⁹L⁻¹. When the count of WBC is less than 4.0×10⁹L⁻¹, immunity of patients is weakened, and the patients are easy to be infected by bacterial and viral, the serious infections will endanger the patient's life. Chronic inflammation in body is also easy to acute attack.

Although human body can recover the number of WBCs, it will take a long period of time. Clinical trial results showed that the WBC level in the placebo group needs 21 days to normal, which greatly increased the risk of infection. It is known that normal number and functions of WBC guaranteed body resistance. A variety of drugs have been used in the treatment of leukopenia, wherein G-CSF is the most famous drugs. However, in the clinical treatment, G-CSF occurred some problems of high clearance rate, short half-life and other issues. PEG modification has provided an excellent solution to the above problems, wherein stability and effect of PEG-modified G-CSF are significantly improved. In clinical treatment, G-CSF 5 μg/kg/day given subcutaneously initiated 24-72 hours after the last day of chemotherapy until sufficient/stable post-nadir WBC recovery. However, pegfilgrastim (PEG-modified G-CSF) given subcutaneously as a single-dose of 100 μg/kg (individualised), is considered as effective as G-CSF. Pegfilgrastim increases the terminal elimination half-life and decreases the apparent serum clearance of the drug in patients, thus it produces a long-acting cytokine to avoid multiple injections. Nevertheless, it has to face a fact that even if the patients receive effective drug treatment, risk of infection cannot be avoided in a certain period of time. Clinical trials showed that after G-CSF treatment, the count of WBC is generally recovers to a normal level with about 1 week. It means the patients are easy to be infected by bacterial and viral during this period, and therefore, it is vitally important to develop a long-lasting and fast-acting drug for patients with leukopenia. In early studies of the inventor, recombinant Ganoderma Lucidum immunoregulatory protein (rLZ-8) was found to recover the number of WBCs effectively in the laboratory animals suffering with leukopenia by different mechanism with G-CSF. Its effect has pointed a new way to treat patients with leukopenia caused by chemotherapy.

rLZ-8 was isolated and identified from a mycelium of ganoderma. A structure of the recombinant Ganoderma Lucidum immunoregulatory protein comprises: an important N-terminal domain for forming a dimer and a C-terminal FNIII domain; wherein the N-terminal domain of the rLZ-8 comprises an α-helix and a β-strand, which forms an important dumbbell-shaped dimer binding domain through space exchanging with the respective domains of another rLZ-8. It has been reported that the rLZ-8 has biological activity on immunoloregulation and killing tumor cells. However, a molecular weight of the dimer is less than 26 kDa; a clearance rate is high; a half-life is short; and pharmacokinetic parameters is difficult to meet requirements of pharmaceutical developments; therefore, only by extending duration of the rLZ-8 in vivo by chemical modification and other technical methods can solid foundations be laid for clinical application.

In the field, the technology of methoxypolyethyleneglycol (mPEG) modification has been developed a primary method to extend half-life of the therapeutic biologics. At present, mPEG's chemical formula is: CH₃O(CH₂CH₂O)nCH₂CH₂OH; one of the terminals is closed by an inert methoxy group in such a manner that molecular crosslinking or agglomeration during modification process can be effectively avoided. As a first generation PEG (polyethyleneglycol)) derivative, PEG-disulfide (PEG-SS for short) has a backbone comprising an ester group which is readily hydrolysable inside a body, and succinate ester fragments left in protein have immunogenicity. As the second generation PEG derivatives, mPEG-succinimidyl propionate (mPEG-SPA for short) and PEG-succinimidyl butyrate (PEG-SBA for short) have no ester group in the backbones in such a manner that a stable connection bond with the protein or polypeptide can be formed, and the mPEG-SPA as well as the PEG-SBA have been widely adapted. However, a modification reaction is a non-directional reaction, and the mPEG-SPA may bind with the respective groups on different sites of the interested protein. If space steric caused by mPEG modification doesn't physiologically affect an active center of the modified protein, then activity of the modified protein is inhibited slightly; if the modification position bind around the active center, then biological activity and physicochemical property of the modified protein may be affected seriously. Meanwhile, modification at different positions will certainly generate a large number of isomers and by-products. The characteristic that single-site modification products exist in the modified medicine as well as multi-site modification products is a main technical bottleneck distressing developments of novel medicine quality researches.

Usually, the protein structures need to be preprocessed for obtaining the specific and identical modification products; the readily modified groups on protein chains are substituted or protected, and then the groups are substituted back or unprotected after the modification reaction, but a period of the reaction is extended and a cost is increased, and the biological activity of the protein medicine may be affected. If conditions can be effectively controlled for selectively providing modification reaction, so as to obtain identical products with the known structures, then safety and controllability of the medicine will be greatly improved and more in line with present guiding principles of novel medicine developments.

### SUMMARY OF THE PRESENT INVENTION

An object of the present invention is to provide an mPEG-SPA modification product of a recombinant Ganoderma Lucidum immunoregulatory protein rLZ-8, a preparation method and applications thereof in preparation of medicine for treating leukopenia due to chemotherapy.

Accordingly, in order to accomplish the above object, the present invention provides the mPEG-SPA modified rLZ-8, wherein the rLZ-8 dimer is single-site modified at an N-terminal; the rLZ-8 dimer binds with the mPEG-SPA, and a molar ratio thereof is 1:1.

A constitutional formula of the mPEG-SPA is as follows:

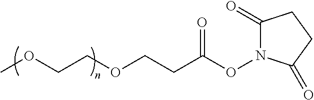

In the constitutional formula, an n value is between 10 and 451, a molecular weight is between 500 Da and 20000 Da.

The present invention also provides the method for preparing the mPEG-SPA modified rLZ-8, comprising steps of:

a) feeding the rLZ-8 dimer and the mPEG-SPA with the molar ratio of 1:1-1:6 into a 0.1M phosphate buffer with pH 5.0-pH 8.0, putting in a penicillin bottle, wrapping with tin foil for being away from light, and stirring by a magnetic stirrer at a room temperature for 1-2.5 h;

b) identifying the product by SDS-PAGE (sodium dodecyl sulfate polyacrylamide gel electrophoresis), staining gel with barium iodide for observing the mPEG-SPA component;

c) purifying and recovering the product, wherein Superdex™75 prep grade chromatography is used for purifying the reaction product, a mobile phase is 0.05M phosphate buffer comprising 0.15M NaCl, the pH is 7.0, a flow rate is 1 mL/min, elution is provided with equivalent concentrations, detection wavelengths are 280 nm, 254 nm, and 215 nm, and the product is collected by a combined method of fixed volume collection and peak collection; and

d) analyzing the purified and recovered samples with the SDS-PAGE, staining the gel with the barium iodide, wherein it is illustrated by identifying with mass spectrometry that the rLZ-8 is only modified at the N-terminal by the PEG, and other sites are not modified; it is further illustrated that the mPEG-SPA modified rLZ-8 obtained in the present invention is the identical modification product; purifying the product for obtaining the modification product with a purity of 98%.

In the present invention, leukopenia treatments are respectively provided on the modified sample and the native sample, and leukocytes are counted by a cytoanalyzer; the results illustrates that significant difference exits between the rLZ-8 and the mPEG-SPA modification product thereof. Wherein, with a same dosage, a period of the mPEG-SPA modified rLZ-8 for promoting growth of the leukocyte is shorter; with a same treatment period, a number of the leukocyte promoted of the mPEG-SPA modified rLZ-8 is larger. Therefore, it is further illustrated that efficiency of the rLZ-8 on treating the leukopenia is significantly enhanced due to the mPEG-SPA modification.

Therefore, the present invention has advantages as follows: the half-life of the the mPEG-SPA modified rLZ-8 provided by the present invention is significantly extended in comparison to the unmodified rLZ-8; the method for preparing the mPEG-SPA modified rLZ-8 is simple, and the product is identical; under conventional conditions, the mPEG readily modifies on lysine residues, and a primary structure of the rLZ-8 has six lysine residues, that is to say, the rLZ-8 dimer have 12 potential sites for modification, and a large variety of the products with the different modified sites may be produced; the present invention obtains the identical products by controlling the reaction conditions without substituting or protecting any groups as well as other extra process; the method is simple and avoids forming a large variety of the products with the different modified sites; pharmaceutical tests in the present invention proves that the half-life of the mPEG-SPA modified rLZ-8 is significantly longer than the half-life of the unmodified rLZ-8; at the same time, the minimum effective dosage and time for treating leucopenia are also improved.

### BRIEF DESCRIPTION OF THE DRAWINGS

FIG. 1 illustrates SDS-PAGE of a purified product after rLZ-8 reacts with mPEG-SPA.

FIG. 2 illustrates tissue distribution of mPEG-SPA modified rLZ-8 and native rLZ-8 in injection position

FIG. 3 illustrates tissue distribution of mPEG-SPA modified rLZ-8 and native rLZ-8 in marrow

FIG. 4 illustrates the distribution of mPEG-SPA modified rLZ-8 and native rLZ-8 in peripheral blood

FIG. 5 illustrates subcellular localization of rLZ-8 on hematopoietic stem cells (A) and T cells (B)

### DETAILED DESCRIPTION OF THE PREFERRED EMBODIMENT

Referring to preferred embodiments, the present invention is further illustrated.

A method for preparing the mPEG-SPA modified rLZ-8 is illustrated with the following preferred embodiments, wherein rLZ-8 is provided by Jilin University, mPEG-SPA is purchased from Shanghai Yarebio Biological Technology Co., Ltd.

The preferred embodiment 1: preparation reactions and product identification of mPEG-SPA modified rLZ-8

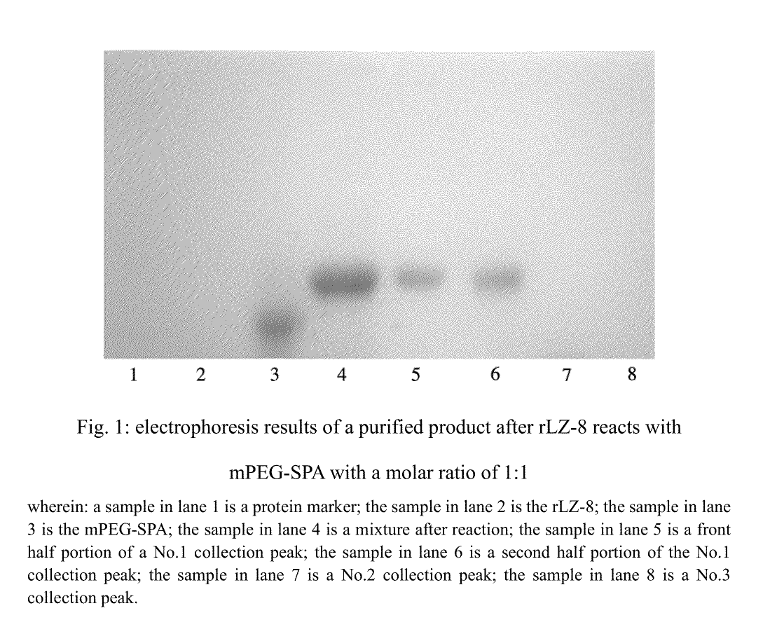

Providing a reaction of the rLZ-8 dimer and the mPEG-SPA (molecular weight is 500 Da) with a molar ratio of 1:1, wherein that is to say, 50 mg the rLZ-8 reacts with 1 mg the mPEG-SPA; wherein a buffer of the reaction is 0.1M phosphate buffer at pH 8.0; avoiding light and stirring, and keeping reacting at a room temperature for 0.5 h; identifying the product by SDS-PAGE, staining gel with barium iodide and analyzing by a gel imaging system, wherein results thereof (referring to FIG. 1) illustrate that under such conditions, bands of the rLZ-8 and mPEG-SPA are decreased, while bands of modified products are widened; and

purifying the product with Superdex™75 prep grade chromatography, wherein the purification condition is: a Superdex™75 prep grade (produced by GE Healthcare) chromatogram column (Type Code: XK16/70) is eluted with equivalent concentrations, wherein a mobile phase is 0.05M phosphate buffer comprising 0.15M NaCl, the pH is 7.0, a flow rate is 1 mL/min, detection wavelengths are 280 nm, 254 nm, and 215 nm; collecting the product by a combined method of fixed volume collection and peak collection, obtaining 40 mg the modification product with a purity of 98%.

According to a mass spectrometry method for identifying modified sites of the mPEG-SPA modified rLZ-8, in the PMF of the modified sample, a matched peptide fragment generally indicates that the peptide is not modified by PEG; sequences of the PEG modified peptide is not matched in the PMF; secondly, the PEG modified site is usually at an N-terminal or a side chain of lysine; furthermore, the PEG modified lysine is generally hard to be digested, therefore, the peptide fragment modified at the lysine by the PEG should comprise at least one omitted site, a molecular weight difference between the PEG modified peptide and the PEG should be approximate to theoretical mass of the peptide fragment, and a mass peak shape of the PEG modified peptide fragment should be basically consistent with the mass peak shape of the PEG.

Results of the detection illustrate that: all the lysine in the PEG modified protein are matched, and it can be judged that the PEG modified sites are not at the lysine; the molecular weight of PEG modified peptide is extremely approximate to the molecular weight of the native PEG, indicating that the PEG modified sites are not at unmatched 75th-111th amino acid; in the PMF of the PEG modified protein, the fragments with and without methionine are detected at the N-terminals of the protein, which may indicate that some of the PEG modified sites are at the N-terminals of the protein, and during a proteolysis process, some of the methionine are split in such a manner that the peptide fragments without the methionine are matched; in addition, the molecular weight difference between the PEG modified peptide and the native PEG is approximate to the molecular weight of the methionine, which further confirms that the PEG modified sites are at the N-terminals of the protein.

The preferred embodiment 2: the preparation reactions and the product identification of the mPEG-SPA modified rLZ-8

Providing the reaction of the rLZ-8 dimer and the mPEG-SPA (the molecular weight is 5000 Da) with a molar ratio of 1:2 in the 0.1M phosphate buffer at pH 7.0, wherein that is to say, 2.5 mg the rLZ-8 reacts with 1 mg the mPEG-SPA; putting the mixture in the penicillin bottle, and keeping reacting at the room temperature for 1 h; identifying and purifying the product, wherein the purifying method is the same as in the preferred embodiment 1, obtaining 2.4 mg the modification product with a purity of 98%; wherein the identifying results are the same as in the preferred embodiment 1.

The preferred embodiment 3: the preparation reactions and the product identification of the mPEG-SPA modified rLZ-8

Providing the reaction of the rLZ-8 dimer and the mPEG-SPA (the molecular weight is 10000 Da) with a molar ratio of 1:4 in the 0.1M phosphate buffer at pH 6.0, wherein that is to say, 5 mg the rLZ-8 reacts with 8 mg the mPEG-SPA; putting the mixture in the penicillin bottle, avoiding light and keeping reacting at the room temperature for 1.5 h; identifying the product by the SDS-PAGE, staining the gel with the barium iodide and analyzing by the gel imaging system, wherein the identifying and purifying methods are the same as in the preferred embodiment 1, obtaining 5.6 mg the modification product with a purity of 98%; wherein the identifying results are the same as in the preferred embodiment 1.

The preferred embodiment 4: the preparation reactions and the product identification of the mPEG-SPA modified rLZ-8

Providing the reaction of the rLZ-8 dimer and the mPEG-SPA (the molecular weight is 20000 Da) with a molar ratio of 1:6 in the 0.1M phosphate buffer at pH 8.0, wherein that is to say, 5 mg the rLZ-8 reacts with 24 mg the mPEG-SPA; putting the mixture in the penicillin bottle, avoiding light and keeping reacting at the room temperature for 2 h; identifying the product by the SDS-PAGE, staining the gel with the barium iodide and analyzing by the gel imaging system, wherein the identifying and purifying methods are the same as in the preferred embodiment 1, obtaining 7.2 mg the modification product with a purity of 98%; wherein the identifying results are the same as in the preferred embodiment 1.

The preferred embodiment 5: effects of the mPEG-SPA modified rLZ-8 on the leukocytes of rats

Utilizing Wistar rats in the experiments, wherein 18 rats weighting about 100 g are utilized. A method for preparing reagents comprises steps of: dissolving the rLZ-8 in sterile saline, and diluting into 60 μg/kg, 30 μg/kg and 15 μg/kg dosage groups; dissolving the mPEG-SPA modified rLZ-8 (the molecular weight is 10000 Da) in the sterile saline, and diluting into 60 μg/kg, 30 μg/kg and 15 μg/kg dosage groups; diluting GenLei®Scimax® [recombinant human granulocyte colony-stimulating factor injection (rhG-CSF)], batch number: 20060403, 75 μg/vial, into 13.5 μg/ml and 0.1 ml per rat with the sterile saline; diluting cyclophosphamide (CP) injection, batch number 050216, 200 mg/vial, into 20 mg/ml and 0.1 ml per rat with the sterile saline, or 20 mg/kg.

The experiment has a normal control group, a low-dosage protein group, a middle-dosage protein group, a high-dosage protein group, a low-dosage mPEG-SPA modified rLZ-8 group, a middle-dosage mPEG-SPA modified rLZ-8 group, a high-dosage mPEG-SPA modified rLZ-8 group, and a positive medicine control group (utilizing the GenLei®Scimax®). The rats of each the group are injected with the cyclophosphamide in tail vein for three days except that the sterile saline is given to the normal control group, the dosage is 20 mg/ml and 0.1 ml per rat. On the third day, the blood is sampled from the tail vein, and the leukocytes are counted by a cytoanalyzer. After successful modeling, the rats of each the group are respectively treated with the rLZ-8, the mPEG-SPA modified rLZ-8, or the positive medicine (the GenLei®Scimax®) with the corresponding dosage, and the equal sterile saline is given to the rats of the normal control and a CP group. The blood is sampled from the tail vein on the first, third and seventh treatment days, and the leukocytes are counted by the cytoanalyzer. Medicine efficacy is analyzed according to a number difference of the leukocyte between before and after the treatment.

It is illustrated in Table 1 that on the first treatment day, the leukocyte number of the rats of the mPEG-SPA modified rLZ-8 groups is significantly increased in comparison with the rats of the CP group, and on the seventh treatment day, the leukocyte number basically approaches to a normal level. On the first treatment day, the leukocyte number of the rats of the mPEG-SPA modified rLZ-8 groups is significantly increased in comparison with the rats of the GenLei®Scimax® control group, and on the seventh treatment day, the leukocyte number basically approaches to the normal level. It is emphasized that when compared with the rLZ-8 groups with the same dosage, the mPEG-SPA modification groups have a sufficient leukocyte proliferation effect from the first treatment day, wherein the leukocyte number is about 2 times more than the leukocyte number of the rLZ-8 groups; with a same treatment period, the leukocyte proliferation effect of the low-dosage modified rLZ-8 group is superior to the leukocyte proliferation effect of the high-dosage rLZ-8 group and other rLZ-8 groups.

TABLE 1
effects of rLZ-8 on rats models with leucopenia (x ± s, n = 10)

| | leukocyte number | on the first | on the third | on the seventh |
|---|---|---|---|---|
| group | before treatment | treatment day | treatment day | treatment day |
| normal | 14.57 ± 2.15 × 10⁹ · L⁻¹ | 13.12 ± 1.75 × 10⁹ · L⁻¹ | 12.8 ± 1.82 × 10⁹ · L⁻¹ | 9.13 ± 2.64 × 10⁹ · L⁻¹ |
| control | | | | |
| CP | 4.9 ± 0.92 × 10⁹ · L⁻¹ | 5.1 ± 1.21 × 10⁹ · L⁻¹ | 5.23 ± 1.34 × 10⁹ · L⁻¹ | 10.27 ± 1.92 × 10⁹ · L⁻¹ |
| control | | | | |
| GenLei ® | 4.37 ± 0.87 × 10⁹ · L⁻¹ | 5.4 ± 0.98 × 10⁹ · L⁻¹ | 10.83 ± 1.23 × 10⁹ · L⁻¹* | 10.17 ± 2.11 × 10⁹ · L⁻¹ |
| Scimax ® | | | | |
| rLZ-8 | 2.93 ± 0.75 × 10⁹ · L⁻¹ | 4.1 ± 0.68 × 10⁹ · L⁻¹ | 8.8 ± 1.53 × 10⁹ · L⁻¹* | 10.2 ± 2.45 × 10⁹ · L⁻¹ |
| (15 μg/kg) | | | | |
| rLZ-8 | 2.96 ± 0.34 × 10⁹ · L⁻¹ | 4.9 ± 0.75 × 10⁹ · L⁻¹ | 9.3 ± 0.88 × 10⁹ · L⁻¹* | 12.83 ± 1.79 × 10⁹ · L⁻¹ |
| (30 μg/kg) | | | | |
| rLZ-8 | 4.33 ± 0.88 × 10⁹ · L⁻¹ | 4.4 ± 1.33 × 10⁹ · L⁻¹ | 8.6 ± 2.01 × 10⁹ · L⁻¹* | 15.5 ± 3.54 × 10⁹ · L⁻¹ |
| (60 μg/kg) | | | | |
| mPEG- | 3.11 ± 1.12 × 10⁹ · L⁻¹ | 8.5 ± 1.22 × 10⁹ · L⁻¹* | 10.8 ± 1.47 × 10⁹ · L⁻¹ | 13.2 ± 2.46 × 10⁹ · L⁻¹ |
| SPA | | | | |
| modified | | | | |
| rLZ-8 | | | | |
| (10 μg/kg) | | | | |
| mPEG- | 2.89 ± 0.65 × 10⁹ · L⁻¹ | 9.2 ± 1.59 × 10⁹ · L⁻¹* | 11.98 ± 1.72 × 10⁹ · L⁻¹ | 13.4 ± 2.54 × 10⁹ · L⁻¹ |
| SPA | | | | |
| modified | | | | |
| rLZ-8 | | | | |
| (30 μg/kg) | | | | |
| mPEG- | 3.33 ± 1.03 × 10⁹ · L⁻¹ | 8.4 ± 1.73 × 10⁹ · L⁻¹* | 14.4 ± 2.01 × 10⁹ · L⁻¹ | 15.5 ± 3.05 × 10⁹ · L⁻¹ |
| SPA | | | | |
| modified | | | | |
| rLZ-8 | | | | |
| (60 μg/kg) | | | | |

In comparison to CP control group,
*p < 0.01

The preferred embodiment 6: tissue distribution contrast experiment of mPEG-SPA modified rLZ-8 in normal rats

¹²⁵I labeling experiment of rLZ-8 and mPEG-SPA-rLZ-8: weighting 1 mg Iodogen (from Sigma) and dissolving in 0.5 mL chloroform, adding 50 μL (100 μg) to a bottom of a test tube, then drying with N₂, and respectively adding 1.5 mL (1.4 mg/mL) rLZ-8 and mPEG-SPA-rLZ-8 for mixing; then adding Na¹²⁵I (wherein specific activity thereof is 107.307 kBc·μg⁻¹) 4 mCi, reacting at 20° C. for 12 min, shaking while reacting for thorough reaction.

Separation and purification of labeled products: purifying ¹²⁵I labeled product with Sephacryl™ S-200 HR, washing with 20 mM pH 7.4 phosphate buffer, automatically collecting one tube of eluant once every 2 min, and totally collection 60 tubes; detecting concentration and radioactivity for each tube.

Content detection of ¹²⁵I-rLZ-8 and ¹²⁵I-mPEG-SPA-rLZ-8: detecting protein concentration with a di-cinchonic acid method, which specifically comprising: adding 10 μL standard bovine serum albumin (2-10 μg) or a sample to each well of a 96-well plate, adding 200 μL protein determination liquid (di-cinchonic acid:4% cupric sulfate solution=1:50) and thoroughly mixing, incubating at 37° C. for 30 min and cooling to a room temperature, detecting an optical absorption value at a wavelength of 562 nm with an enzyme labeling machine, adjusting with water; obtaining a net protein absorption value by each absorption value minus blank tube absorption value, then drawing a standard curve and calculating a protein content with linear regression.

Tissue distribution experiment: randomly dividing 72 Wistar rats into 2 groups (Native rlZ-8 group and mPEG-SPA-rLZ-8 group) with 36 rats in each group, 18 males and 18 females, whose weights are 180±20 g; subcutaneously injecting the rats with ¹²⁵I-mPEG-SPA-rLZ-8 and ¹²⁵I-rLZ-8 once with a dosage of 125 μg/kg; respectively taking 6 rats at different time points (6 h, 12 h, 24 h, 48 h, 72 h and 96 h) (3 males and 3 females) for anatomy, wherein the rats are respectively injected at partial subcutaneous tissue, bone marrow and peripheral blood, weighting and cutting into pieces, adding 400 μL 20% TCA for protein precipitation, and detecting a total γ radiation; after centrifugation, respectively detecting γ radiation of the precipitation and supernatant; wherein drug concentration is represented by equivalent concentration (ng Equ g⁻¹).

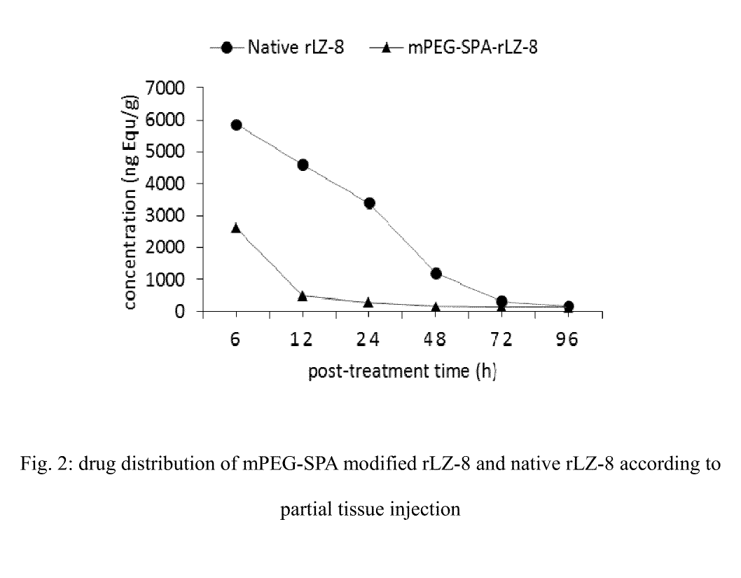

Referring to FIG. 2, in the first 48 h after feeding the rLZ-8, drug concentration at partial tissue injected is high; while after 12 h, concentration of the mPEG-SPA modified rLZ-8 after partial tissue injected is reduced to a lower level, which illustrates that the rlz-8 residents at the partial tissue injected, and mPEG-SPA-rLZ-8 has sufficient dispersion effect. rLZ-8 comes from fungi, so it lacks corresponding receptor for transporting it into peripheral blood in Mammals. The results show that the rlz-8 will resident at the partial tissue after injection (referring to FIG. 2). The mPEG-SPA modification is able to improve the phenomenon because mPEG modification changes physical properties of the rLZ-8, and improves dispersion rate for reducing partial resident time.

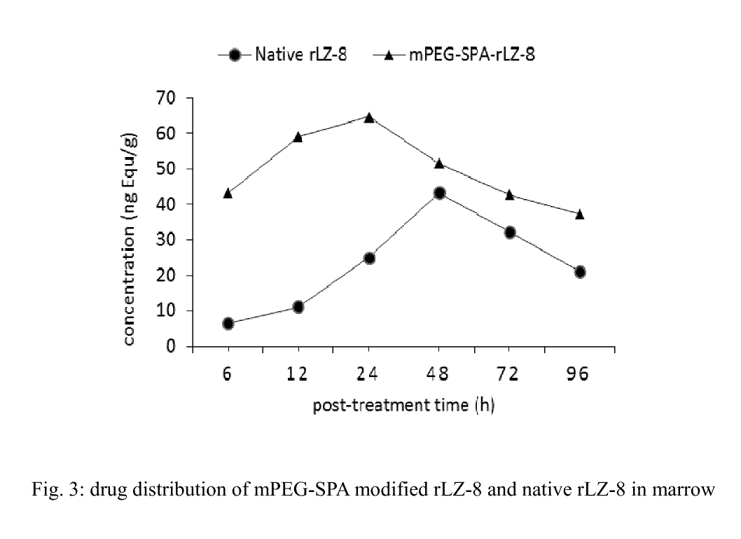

Bone marrow is one of the major target organs in treating leucopenia. Referring to FIG. 3, drug concentration of rLZ-8 in bone marrow reaches a peak 48 h after injection, and drug concentration of mPEG-SPA-rLZ-8 in bone marrow reaches a peak 12-24 h after injection. It can be concluded that the mPEG-SPA-rLZ-8 distributes to the target organs faster than the rLZ-8 does, and thus plays a role more rapidly. According to table 1, on the first day of treatment, white blood cell level of the mPEG-SPA-rLZ-8 is rapidly increased, which is significantly higher than that of the rLZ-8 group. The results coincide with experimental data of drug metabolic tissue distribution of rLZ-8 and mPEG-SPA-rLZ-8.

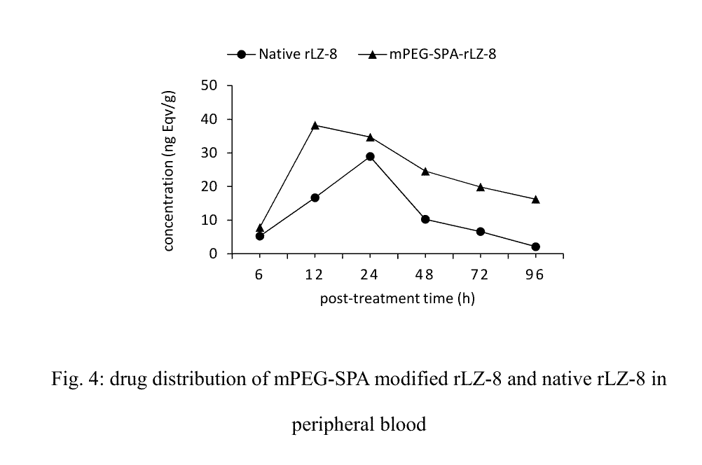

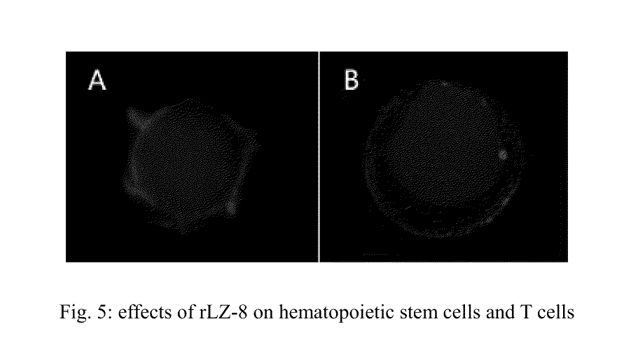

Referring to FIG. 4, distribution of the mPEG-SPA-rLZ-8 and the rLZ-8 in peripheral blood is shown, wherein concentration of the mPEG-SPA-rLZ-8 in the peripheral blood reaches a peak 12 h after injection, while concentration of the rLZ-8 reaches a peak 24 h after injection. The drug concentration in the peripheral blood reaches the peak earlier than the one in the bone marrow does, but the drug concentration in the peripheral blood is lower than the one in bone marrow. However, the rLZ-8 is in a dimer form with a molecular weight of 26 kDa. Research has proved that when a molecular weight is more than 16 kDa, drug subcutaneously injected is mainly distributed through the lymphatic system. Furthermore, the rLZ-8 lacks specific transport receptor in vivo, and rLZ-8 is able to be combined with a variety of immune cells such as T cells (referring to FIG. 5). Therefore, a number of the rLZ-8 which is transported to the peripheral blood through the lymphatic system is small, which does not affect tissue distribution thereof.

Effects of the rLZ-8 on cells of an immune system and a hematopoietic system are respectively studied with T cells and hematopoietic stem cells as examples, wherein experiment is as follows:

using BALB/c mice as experimental animals, separating mouse hematopoietic stem cells with STEMCELL EasySep™ hematopoietic stem cell isolation kit (product number #19856), separating T cells with Life Technologies Dynabeads® FlowComp™ Mouse Pan T (product number #11465D). fluorescent-labeling rLZ-8 protein with Life Technologies Alexa Fluor® 568 Protein Labeling Kit (product number #A10238), treating the hematopoietic stem cells with labeled products (rLZ-8 with a concentration of 10 μg/mL) for 1 h, adding Life Technologies Hoechst 33258 nucleic acid stain (product number #H3569) until the concentration is 1.0 μg/mL, staining for 15 min before slicing; treating the T cells with the labeled products (rLZ-8 with a concentration of 1 μg/mL) for 3 h, adding Life Technologies Hoechst 33258 nucleic acid stain (product number #H3569) until the concentration is 1.0 μg/mL; imaging with GE Delta Vision OMX ultra high resolution microscope; wherein results show that the rLZ-8 is able to be combined with surface receptors of the hematopoietic stem cells and the T cells (referring to FIG. 5), and the rLZ-8 is able to increase the number of white blood cells in the experimental animals by activating the hematopoietic stem cells. A large number of the rLZ-8 is combined with the T cells, which illustrates that lymphokinesis is an important way to transport the rLZ-8 in vivo, and the rLZ-8 is able to affect the hematopoietic stem cells in the bone marrow through lymphokinesis.

What is claimed is:

1. Methoxypolyethyleneglycol succinimidyl propionate modified recombinant Ganoderma Lucidum immunoregulatory protein rLZ-8, wherein said rLZ-8 dimer is single-site modified at an N-terminal.

2. A method for treating leukopenia due to chemotherapy in a subject, comprising: applying a therapeutically effective amount of the mPEG-SPA modified rLZ-8 as recited in claim 1, or a medicinally acceptable salt thereof, to the subject.
